(19) United States
(12) Patent Application Publication
(10) Pub. No.: US 2003/0031688 A1
(43) Pub. Date: Feb. 13, 2003

(54) Cosmetic composition with improved skin moisturizing properties

(76) Inventors: Dipak Ghosh, Woodridge, IL (US); Usha V. Murthy, Oak Brook, IL (US); Munawar Hussain Randhawa, Naperville, IL (US); Natalya Gurman, Mount Prospect, IL (US); Perry Romanowski, Oak Park, IL (US); Medardo Hernandez, Westchester, IL (US); Deborah A. Eagan, Melrose Park, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 09/851,882

(22) Filed: May 9, 2001

Related U.S. Application Data
(60) Provisional application No. 60/281,039, filed on Apr. 2, 2001.

(51) Int. Cl. A61K 38/43; A61K 7/00

(52) U.S. Cl. 424/401; 424/94.1

(57) ABSTRACT

The invention provides a cosmetic composition with improved moisturizing properties, improved skin feel and/or improved visual appeal, comprising a synergistic combination of coenzyme Q₁₀ and urea. The coenzyme Q₁₀ and urea act synergistically to confer improved, cosmetically desirable properties with respect to the composition of the invention.

### CROSS-REFERENCE TO RELATED PATENT APPLICATIONS

The present application claims priority to U.S. provisional patent application No. 60/281,039, filed on Apr. 2, 2001.

### TECHNICAL FIELD OF THE INVENTION

The present invention relates to a topical cosmetic composition that exhibits improved moisturizing properties, as well as to methods related thereto.

### BACKGROUND OF THE INVENTION

Many cosmetic products have moisturizing properties in order to combat drying of the skin. Some moisturizers contain ingredients, such as, for example, gycerin, propylene glycol (PG), hyaluronic acid salts (e.g., sodium hyaluronate), and the like, that provide superficial effects by forming a layer on the surface of the skin. Such layer-forming moisturizers serve as humectants and attract ambient moisture from the atmosphere and trap it onto the skin.

Other moisturizers are designed to work at the cellular level by rebuilding or preventing damage to the skin's natural barrier layer. In this respect, the outermost layer of the epidermis, the horny cell layer (i.e., the stratum corneum), provides a barrier function to protect the skin from ultraviolet light and other environmental influences, and to avoid excessive dryness. The horny cell layer is continually worn down due to contact with the environment and, therefore, must be constantly renewed. However, the regular renewal of the horny cell layer can be interrupted by various endogenous and exogenous factors. As a result, the barrier layer's ability to function properly is impeded because dead, dull skin cells undesirably do not “slough off” (detach or separate) regularly, and the natural lipids in the horny cell layer, such as ceramides, cholesterol, esters and the like, cannot properly control the amount of moisture evaporating from the epidermis. In addition, the natural lipids in the horny cell layer can be damaged from ultraviolet light rays. In particular, ultraviolet light rays can initiate a chain reaction that transforms lipids in the horny cell layer into free radical species. The free radical species, once formed, attack other lipids in the horny cell layer, thereby forming more free radical species which, in turn, results in the damage of more lipids. As more lipids are damaged, the skin's natural moisture barrier loses its ability to effectively regulate moisture retention in the skin.

As the natural lipid layer is impeded or damaged, more moisture escapes from the skin than is absorbed into the skin, such that dry skin results. In response, moisturizers acting at the cellular level are designed to penetrate the surface of the skin and repair the natural lipid layer, for example, by facilitating the ability of dead, dull skin cells to slough off more regularly and/or by acting as free radical scavengers to prevent the free radicals from continuing the deleterious chain reaction.

Despite the availability of conventional moisturizing compositions, there is a continuing effort to make many existing moisturizing cosmetic products more effective by enhancing the amount of water absorbed into the skin, by reducing the amount of endogenous moisture that escapes from the skin, and/or by enhancing the penetration of the composition so that it can act at the cellular level. Previous attempts to enhance the moisturizing benefits of such products have not been fully satisfactory. For example, it has proven to be difficult in practice to provide long lasting moisturization with compositions having ingredients that form a layer on the surface of the skin to trap moisture from the atmosphere onto the skin surface, while at the same time permitting penetration of the moisturizer through the skin surface so that it also can act at the cellular level. There is also a continuing effort to make such cosmetic moisturizing compositions with enhanced “after-feel,” for example, such that they do not render the skin greasy, and so that they have an improved visual appeal.

Accordingly, there is a need in the art for a cosmetic composition with enhanced moisturizing properties, improved after-feel, and/or improved visual appeal. It will be appreciated also that there is a need in the art for a method of enhancing moisture retention in the skin and/or of reducing the rate of escape of water from the epidermis, as well as a method of improving the after-feel of a cosmetic composition when the composition is applied to the skin.

### BRIEF SUMMARY OF THE INVENTION

The present invention provides a cosmetic composition with improved moisturizing properties, e.g., by promoting enhanced moisture retention in the skin or by reducing the rate of escape of water from the epidermis. The composition of the present invention also has improved after-feel when the composition is applied to the skin.

It has been found that urea and coenzyme Q10, when combined in a ratio of urea to coenzyme Q10 of from about 1:5 to about 20:1 (wt./wt.), act synergistically to exhibit significantly improved moisturizing properties, after-feel (e.g., non-greasiness, rapid absorption, and the like), and/or visual appeal. Thus, in one aspect, the present invention provides a cosmetic composition that comprises urea and coenzyme Q10, wherein the ratio of urea to coenzyme Q10 in the composition is from about 1:5 to about 20:1 (wt./wt.).

In another aspect, the present invention provides a method of enhancing moisture retention in the skin or of reducing the rate of escape of water from the epidermis. The method comprises topically applying a moisture-retaining or moisture-loss-inhibiting effective amount of a composition comprising urea and coenzyme Q10 to the skin. The ratio of urea to coenzyme Q10 in the composition is from about 1:5 to out 20:1 (wt./wt.).

In yet another aspect, the present invention provides a method of improving the after-feel of a cosmetic composition, when the composition is applied to the skin. The method comprises adding to or including in the composition an after-feel enhancing-effective amount of coenzyme Q10 and urea, wherein the ratio of urea to coenzyme Q10 is from about 1:5 to about 20:1 (wt./wt.).

The combination of coenzyme Q10 and urea, in accordance with the present invention, synergistically provides unexpected improvements in certain cosmetically desirable properties. In particular, when coenzyme Q10 and urea are combined, there are significant and unexpected improvements in skin moisturizing properties, rate of absorption into the skin, after-feel when applied to the skin, and even in the product's visual appearance. The improvement in these properties is not obtainable when either ingredient is used alone, and the improvement would not have been expected based on the properties of each ingredient, individually.

The present invention may be best understood with reference to the following detailed description of the preferred embodiments in conjunction with the accompanying drawings.

### BRIEF DESCRIPTION OF THE DRAWINGS

FIG. 1 graphically depicts the moisture uptake properties of a synergistic coenzyme Q10/urea blend as compared to coenzyme Q10 and urea, individually.

FIG. 2 graphically depicts the moisturizing properties of a synergistic coenzyme Q10/urea blend as compared to coenzyme Q10 and urea, individually.

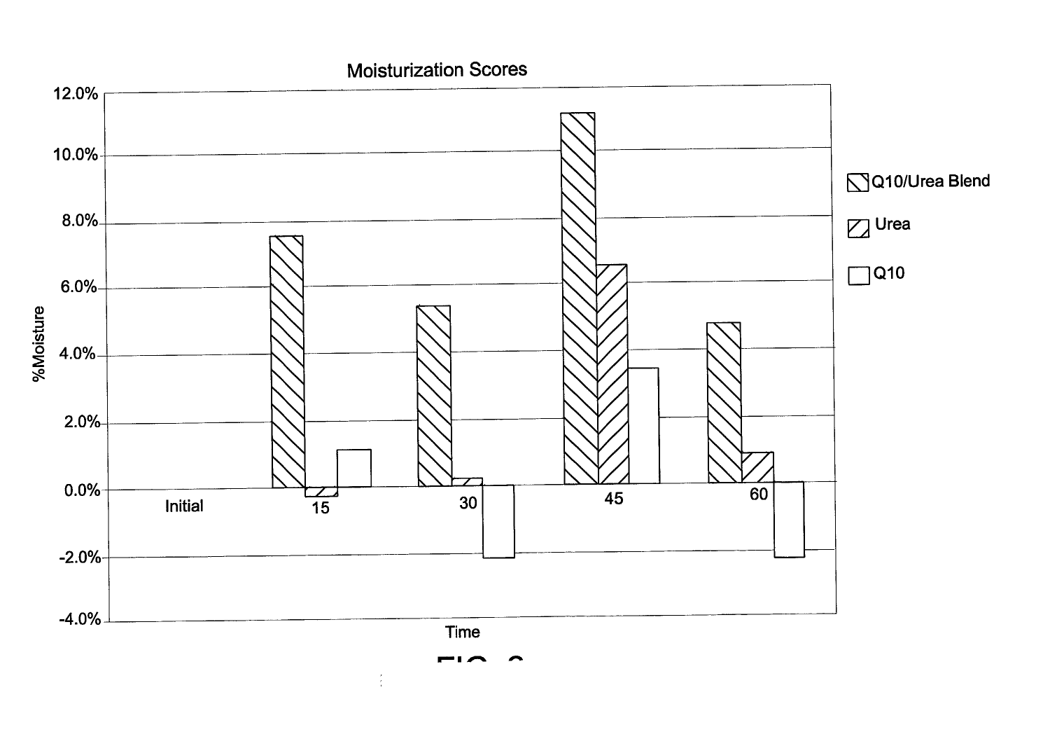

FIG. 3 graphically depicts a comparison of the cumulative moisturizing properties of a coenzyme Q10/urea blend as compared to coenzyme Q10 and urea, individually.

### DETAILED DESCRIPTION OF THE INVENTION

The present invention is predicated, at least in part, on the surprising and unexpected discovery that a cosmetic composition comprising urea and coenzyme Q10, wherein the ratio of urea to coenzyme Q10 in the composition is from about 1:5 to about 20:1 (wt./wt.), exhibits improved moisturizing properties, improved skin feel, and/or improved visual appeal. In this respect, the combination of coenzyme Q10 and urea, according to the present invention, synergistically confers improved cosmetically desirable properties. The moisturizing properties, after-feel, and visual appeal exhibited by the synergistic combination of the present invention are improved over the properties conferred by either ingredient alone, and the improvement would not have been expected based on the properties of each ingredient, individually.

In view of the above, the present invention provides a cosmetic composition comprising urea and coenzyme Q10, wherein the ratio of urea to coenzyme Q 10 in the composition is preferably from about 1:5 to about 20:1 (wt./wt.), as well as methods related thereto. In preferred embodiments, the amount of urea in the composition exceeds that of coenzyme Q10. Preferably, the ratio of urea to coenzyme Q10 in the composition is at least about 3:1, more preferably, at least about 6:1, and even more preferably, at least about 9:1. In some embodiments, the ratio of urea to coenzyme Q 10 is at least about 12:1, such as for example, at least about 15:1, at least about 18:1, at least about 20:1, or even higher. The precise amounts of the urea and the coenzyme Q10 can vary but preferably are selected so as to fall within the aforementioned ratio ranges. Preferably, the urea is present in an amount of from about 0.02% to about 15% by weight of the composition, more preferably from about 0.1% to about 10% by weight of the composition, even more preferably from about 0.1% to about 5% by weight of the composition, e.g., from about 0.1-4%, from about 0.3-4%, from about 0.3-2%, from about 0.3-1%, or from about 0.3-0.5% by weight of the composition. The coenzyme Q10 preferably is present in an amount of from about 0.001% to about 5% by weight of the composition, more preferably, in an amount of from about 0.01% to about 2% by weight of the composition, even more preferably from about 0.02% to about 2% by weight of the composition, e.g., from about 0.02-1.5%, from about 0.02-1%, from about 0.02-0.5%, or from about 0.02-0.05% by weight of the composition.

Coenzyme Q10, also known as ubiquinone 50 and ubidecarenone, is one of the coenzymes Q (“ubiquinones”). The coenzymes Q are naturally occurring in the majority of aerobic organisms, from bacteria to higher plants and animals, and define a group of lipid-soluble benzoquinones involved in electron transport in mitochondria (i.e., in the oxidation of succinate or reduced NADH via the cytochrome system). The ubiquinones all share the 2,3-dimethoxy-5-methylbenzoquinone nucleus but differ in terms of the terpenoid side chain substituted on the remaining carbon atom. The terpenoid side chain comprises from 1-12 mono-unsaturated trans isoprene units.

The individual coenzymes Q are identified by two nomenclatures: “coenzyme Qₙ” and “ubiquinone x,” wherein n is the number of isoprene units in the terpenoid side chain and x is the number of carbon atoms in the terpenoid side chain. Coenzyme Q10 is the most common of the coenzymes Q and has the formula:

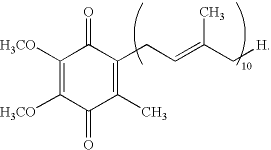

Urea, or carbamide, the diamide of carbonic acid, is a well-known naturally occurring degradation product of proteins in humans. As such, it is found endogenously in body organs, tissues, and body fluids. Urea is proven to be safe for topical application and has been used, e.g., in the treatment of eczema. In accordance with the present invention, the combination of urea and coenzyme Q10 synergistically enhances water absorption. Coenzyme Q10 is a hydrophobic material and it exhibits very poor water absorption capability when used alone. Urea, which is hydrophilic relative to coenzyme Q10, has a higher water absorption property than coenzyme Q10. The combination of coenzyme Q10 and urea in accordance with the present invention, however, exhibits higher water absorption than when either ingredient is used in the composition alone and provides greater water absorption than would have been expected by combining the ingredients. While not wishing to be bound by any particular theory, it is believed that the urea and the coenzyme Q 10 form a complex that has the ability to penetrate the skin so that one or both of the ingredients can function at the cellular level.

The beneficial effects provided by the composition and methods of the invention are useful for a number of different cosmetic applications. The present inventive composition, for example, can be embodied in a variety of products, such as, but not limited to, facial moisturizers, eye creams and lotions, hand renewal lotions, and the like. Furthermore, the composition can be formulated into any suitable form, such as, for example, a gel, a lotion, a cream, a solution, or the like, as will be appreciated by the ordinarily skilled artisan.

In some embodiments, for example, cream and lotion formulations, the composition is in the form of a water-in-oil emulsion or an oil-in-water emulsion. In a water-in-oil emulsion, the oil phase is the continuous (or external) phase, and dispersed within the oil phase is the aqueous (or internal) phase. In an oil-in-water emulsion, the aqueous phase is the continuous phase and the oil phase is dispersed within the aqueous phase. Emulsion formulations are desirable in accordance with preferred embodiments of the invention as they permit uniform application to the skin and provide good coverage, and skin feel.

Even if the composition is not in the form of an emulsion, water and/or oil can be included in the composition. If water and/or oil are included, in an emulsion or otherwise, they can be included in any suitable amount. For example, in emulsion formulations according to some embodiments of the invention, water and oil can be provided in sufficient amounts to serve as the continuous or disperse phase. If present in an emulsion or otherwise, water preferably is present in an amount of from about 50% to about 90% by weight of the composition, and/or oil preferably is present in an amount of from about 10% to about 95% by weight of the composition, more preferably, in an amount of from about 10% to about 50% by weight of the composition.

If present in an emulsion or otherwise, the oil can be in any suitable form. Preferably, oil has emollient properties desirable for moisturizing compositions, as will be appreciated by the ordinarily skilled artisan. For example, in some embodiments, the oil can be selected from silicone oils, triglyceride esters, natural oils, waxes, hydrocarbons, phospholipids, polyhydric and fatty alcohols, polyether derivatives, and the like, or combinations thereof. Suitable oils, as will be appreciated by one of ordinary skill in the art, are described, for example, in U.S. Pat. Nos. 5,980,921 and 6,042,815. Preferred oils include, for example, silicone oils. Silicone oils can be in the form of one or more volatile silicones, non-volatile silicones, and mixtures thereof. Exemplary silicone oils include, for example, cyclomethicone, phenyl trimethicone, alkyl dimethicone, fluorinated silicones, dimethicone, and the like, or combinations thereof.

In emulsion embodiments, the composition also desirably includes an emulsifier. For example, the emulsifier can be included to stabilize the disperse phase (e.g., the aqueous phase in a water-in-oil emulsion or the oil phase in an oil-in-water emulsion) within the continuous phase (e.g., the aqueous phase in an oil-in-water emulsion or the oil phase in a water-in-oil emulsion). As will be appreciated by the ordinary artisan, the emulsifier is selected to be compatible with the other components in the composition, for example, the disperse phase in the case of an emulsion. Any of a number of nonionic, anionic, or cationic emulsifiers can be utilized. Suitable emulsifiers are disclosed, for example, in U.S. Pat. Nos. 3,795,560 and 4,421,769, as well as McCutcheon's Detergents and Emulsifiers, North American Edition, pages 317-324 (1986). By way of example, dicetyl phosphate, ceteth-10-phosphate, sodium stearate, stearic acid, behentrimonium methosulfate, and the like, or combinations thereof can be used as emulsifiers. The emulsifier is preferably present in an amount sufficient to emulsify the disperse phase within the continuous phase of an emulsion. For example, the emulsifier can be present in an amount of from about 1.5% to about 20% by weight of the composition.

Although not essential, the composition of the invention also can include one or more optional ingredients, such as, for example, a thickener, a sunscreen, a preservative, a solid emollient, a soothing additive, an antioxidant other than coenzyme Q10, a chemical neutralizer, an odor masking agent, a moisturizing additive, or the like. Any suitable thickener can be utilized in accordance with the present invention. By way of example, the thickener can be in the form of gum, cellulosics, acrylic polymers, carbomers, and the like, or combinations thereof. Especially suitable thickeners include, but are not limited to, glyceryl stearate, carbomer, C₁₀-C₃₀ alkyl acrylate cross polymer, and the like, or combinations thereof. The thickener can be provided in any suitable amount, for example, to achieve the desired viscosity of the composition. Preferably, the thickener is present in an amount of from about 0.01% to about 5% by weight of the composition.

A sunscreen agent can be optionally included in some embodiments. Suitable sunscreens that are approved for human use by the appropriate regulatory authority of a particular country are preferred and are well known to those of ordinary skill in the art. In the United States, as an example, suitable sunscreens approved by the U.S. Food and Drug Administration (FDA) are described in the “FDA Sunscreen, Final Monograph,” as published in the Federal Register in Jun. 2000 (Vol. 65, No. 111, Pages 36319-36324). By way of example, suitable sunscreens include octyl methoxycinnamate, oxybenzone, ethylhexyl p-methoxycinnamate, and the like, or combinations thereof. If included, the sunscreen can be present in any suitable amount, such as, for example, in an amount of from about 0.01% to about 20% by weight of the composition.

In addition, the composition optionally can include a preservative. For example, the preservative can be selected to kill bacteria that might otherwise be sustained or multiply in the composition. Such preservatives are well known to those of ordinary skill in the art and examples of suitable preservatives will be appreciated by the ordinarily skilled artisan. In this respect, the type of preservative chosen will depend upon the components and the structure of the composition. For example, some preservatives are selected to combat microorganisms that are sustained in water, while others are selected to combat microorganisms that are sustained in oil, as will be appreciated by the ordinarily skilled artisan. Exemplary preservatives include, but are not limited to ethylparaben, propylparaben, methylparaben, EDTA or salts thereof, phenoxyethanol, DMDM hydantoin, and the like, or combinations thereof. The preservative is present in any suitable amount, such as an amount of from about 0.01% to about 3% by weight of the composition.

Optionally, the composition can include a solid emollient. The solid emollient desirably can be included to complement the emollient properties of the oil, if present. Any suitable solid emollient can be selected, as will be appreciated by one of ordinary skill in the art. By way of example, the solid emollient can be selected from C₁₂-C₁₅ alkyl benzoate, C₁₂-C₁₅ alkyl octanoate, and the like, or combinations thereof. The solid emollient can be included in any suitable amount, such as, for example, in an amount of from about 0.1% to about 10% by weight of the composition.

In some embodiments, the composition optionally can include one or more soothing and/or moisturizing additives. Any suitable soothing and/or moisturizing additive can be selected. Exemplary soothing additives include, but are not limited to, allantoin, natural moisturizing and soothing botanical extracts, anti-inflammatory agents, and the like, or combinations thereof. Exemplary moisturizing additives include, but are not limited to, esters, humectants, natural botanical extracts, such as chamomile recutita extract, sambucus nigra extract, primula veris extract, glycerin, helianthus annuus extract and the like, as well as phospholipids, silicones, occlusive agents, natural oils, barbadensis gel, and the like, or combinations thereof. If included, the soothing and/or moisturizing additives can be included in any suitable amount. For example, the soothing additive can be present in an amount of from about 0.001% to about 5% by weight of the composition, and the moisturizing additive can be present in an amount of from about 0.0001% to about 25% by weight of the composition.

To complement the oxidative properties of the coenzyme Q10, any suitable antioxidant other than coenzyme Q10 optionally can be included in the composition. Such antioxidants can include antioxidant vitamins, such as, for example, Vitamins C, and E, as well as, for example, superoxide dismutase (SOD), ascorbic acid, and the like, or combinations thereof. The antioxidant other than coenzyme Q10 can be included in the present inventive composition in any suitable amount, such as, for example, in an amount of from about 0.001% to about 5% by weight of said composition.

If desired, the composition optionally can include one or more chemical neutralizers, such as, for example, strong and weak bases. Any suitable chemical neutralizer can be selected, as will be appreciated by one of ordinary skill in the art. The neutralizer can be selected, for example, from sodium hydroxide, potassium hydroxide, ammonium hydroxide, diethanolamine, triethanolamine, 2-dimethylamino-2-methyl-1-propanol (DAMP), 2-amino-methyl-1 propanol (aminomethyl propanol) (AMP), and the like, or combinations thereof. The chemical neutralizer can be provided in any amount, e.g., an amount sufficient to achieve a desired pH for the composition. In this respect, the composition preferably has a pH of from about 2-12, more preferably, from about 4-9, and still more preferably, from about 5-8. Preferably, the chemical base is present in an amount of from about 0.01% -10% by weight of the composition.

To enhance the aesthetics of the composition, any suitable odor-masking agent optionally can be included. As will be appreciated by one of ordinary skill in the art, suitable odor masking agents can be selected from various natural and synthetic sources. A preferred odor-masking agent is ethylene brassylate. The odor masking agent can be present in any suitable amount, such as, for example, an amount of from about 0.01% to about 3% by weight of the composition.

In some embodiments, the composition is in the form of a water-in-oil emulsion or an oil-in-water emulsion comprising an aqueous phase, which preferably is present in an amount of from about 50% to about 90% by weight of the composition; an oil phase, which preferably is present in an amount of from about 10% to about 50% by weight of the composition; an emulsifier, which preferably is present in an amount of from about 0% to about 20% by weight of the composition; a thickener, which preferably is present in an amount of from about 0% to about 5% by weight of the composition; a preservative, which preferably is present in an amount of from about 0.01% to about 3% by weight of the composition; a sunscreen, which preferably is present in an amount of from about 0% to about 20% by weight of the composition; a chemical neutralizer, which preferably is present in an amount of from about 0% to about 10% by weight of the composition; an odor-masking agent, which preferably is present in an amount of from about 0% to about 3% by weight of the composition; a solid emollient, which preferably is present in an amount of from about 0% to about 10% by weight of the composition; a soothing additive, which preferably is present in an amount of from about 0% to about 5% by weight of the composition; a moisturizing additive, which preferably is present in an amount of from about 0% to about 20% by weight of the composition; and an antioxidant other than Q 10, which preferably is present in an amount of from about 0% to about 1% by weight of the composition.

In accordance with the present invention, upon topical application of the composition to the skin, the composition preferably imparts an increase in water absorption as measured by moisture index (Novameter DPM 9003) of at least about 50% relative to untreated skin when applied in an amount of 155 mg/cm² skin about 120 minutes after application, more preferably an increase of at least about 55% relative to untreated skin when applied in an amount of 155 mg/cm² skin about 120 minutes after application, still more preferably, an increase of at least about 60% relative to untreated skin when applied in an amount of 155 mg/cm² skin about 120 minutes after application. It is also preferred that the composition of the present invention imparts an increase in water absorption as measured by moisture index (Novameter DPM 9003) of at least about 65% relative to untreated skin when applied in an amount of 155 mg/cm² skin about 180 minutes after application, more preferably, an increase of at least about 70% relative to untreated skin when applied in an amount of 155 mg/cm² skin about 180 minutes after application, even more preferably, an increase of at least about 75% relative to untreated skin when applied in an amount of 155 mg/cm² skin about 180 minutes after application, and still more preferably, an increase of at least about 80% relative to untreated skin when applied in an amount of 155 mg/cm² skin about 180 minutes after application. It will be appreciated that the Novameter DPM 9003 is a dermatological laboratory instrument commonly used in the cosmetics industry. In particular, the DPM Series 9000 instruments employ an impedance based electronic sensing system to evaluate biophysical properties of the skin. The scale of the DPM 9003 instrument operates between 90 for a low reading and 999 for a high reading. Lower values generally represent dry skin while higher values represent hydrated or topically treated skin. It will be appreciated that the conversion of Novameter readings into percentage increase in moisturization is well known in the cosmetic industry, and is calculated based on the readings obtained from the Novameter DPM 9003 according to the following equation: Percentage Increase=[(Final Reading−Initial Reading)/Initial Reading]×100.

In using the DPM 9003 instrument, a remote, uniform-pressure sensor probe is employed. Readings are taken by lacing the sensor probe on the surface of the skin. An automatic sensor switch activates the system. The remote sensor port allows the operator to quickly change probes. DPM readings are immediately displayed on the LCD screen located on top of the instrument. DPM measurements represent relative values of skin characteristics based on the capacitive reactance of the skin. DPM readings can be monitored and stored when the optional DPM 9900 series of software is used with an MS-DOS compatible computer.

The following examples further illustrate the invention but should not be construed as in any way limiting its scope.

### EXAMPLE 1

This example demonstrates the water absorption properties of a synergistic combination of coenzyme Q10 and urea. A mixture of urea and coenzyme Q10 (9:1 Wt./wt.)) was prepared as a dry blend of the raw materials by manually mixing urea (9 parts by weight) and coenzyme Q10 (1 part by weight), with mortar and pestle grinding as necessary, to provide an essentially homogeneous powder. The moisture-absorbing properties were determined based on the amount of moisture uptake over time. For comparison, the moisture-absorbing properties of powder samples of the unblended individual raw materials, i.e., urea alone and coenzyme Q10 alone, were measured.

The samples were placed in a controlled humidity chamber (65% relative humidity at 25° C.) and the mass of each sample was measured at various time points. The water uptake is reflected by the increase in mass over time.

The results are shown in Table 1. The percentage values shown in Table 1 reflect the percentage increase in mass relative to the weight of the starting sample (prior to being placed in the controlled humidity chamber). The expected percentage values for the blend (“Expected”) were calculated on the basis of the fractional contribution of each component alone, as represented by the following equation: Expected %=(0.9×% measured for urea alone)+(0.1×% measured for Q10 alone).

TABLE 1

| Averages | Initial | 5 | 10 | 15 | 30 | 45 | 1 hr |
|---|---|---|---|---|---|---|---|
| Urea | 0.0% | 31.0% | 4.0% | 5.5% | 10.6% | 16.6% | 22.4% |
| Q10 | 0.0% | 1.0% | 0.1% | 0.5% | 1.5% | 2.4% | 3.1% |
| Urea/Q10 | 0.0% | 3.9% | 4.8% | 7.3% | 13.4% | 19.8% | 26.3% |
| (9 to 1 | | | | | | | |
| ratio) | | | | | | | |
| Expected | 0.00% | 2.8% | 3.6% | 5.0% | 9.7% | 15.2% | 20.5% |

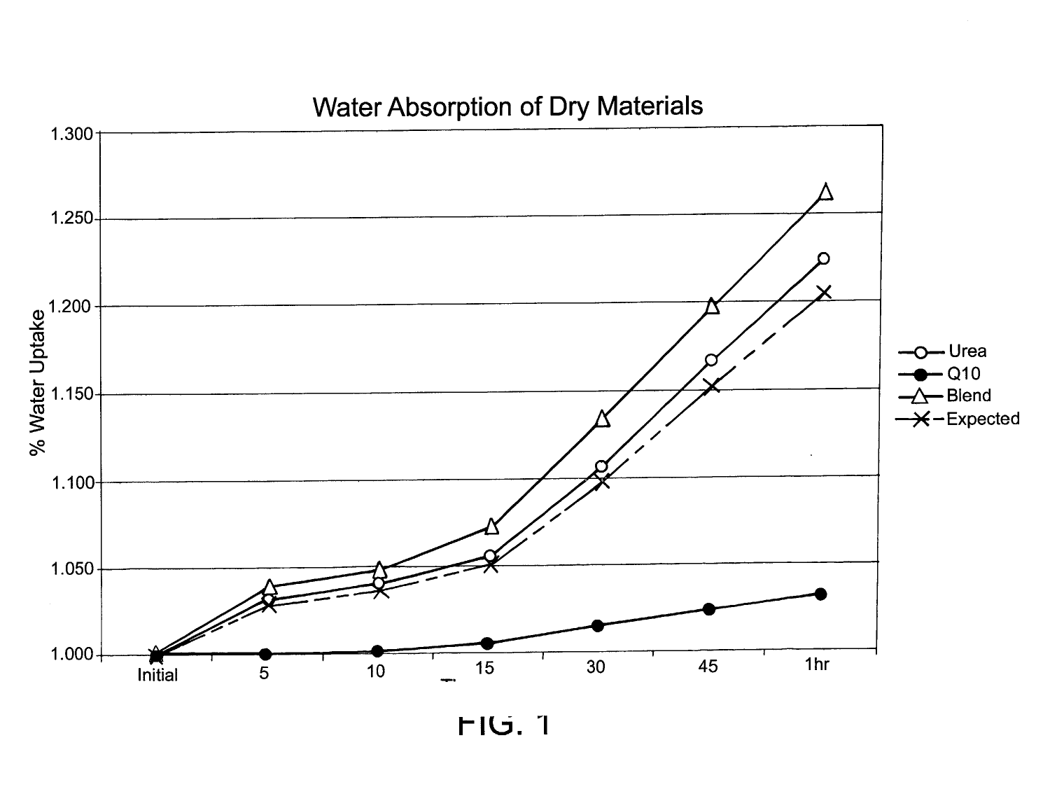

The results in Table 1 are graphically depicted in FIG. 1. These data demonstrate that the coenzyme Q10/urea blend of dry raw materials absorbs more water from the environment than expected (about 6% greater than expected moisture uptake after 1 hour).

### EXAMPLE 2

This example demonstrates the immediate skin moisturizing properties of a synergistic combination of coenzyme Q10 and urea. Three samples (Sample 2A, and Comparative Samples 2B and 2C) were prepared and evaluated. Sample 2A contains a synergistic combination of coenzyme Q10 and urea. Comparative Sample 2B contains coenzyme Q10 in the absence of urea. Comparative Sample 2C contains urea in the absence of coenzyme Q10. Each sample was prepared as an aqueous dispersion containing polysorbate 20 (a nonionic surfactant) and phenoxyethanol (a preservative/antimicrobial agent).

Sample 2A was prepared by dissolving urea and coenzyme Q10 in a standard aqueous polysorbate 20 solution, diluting the resulting solution with water to form a dispersion of urea and coenzyme Q10, and adding 2-phenoxyethanol. The resulting dispersion contained the following components, shown in percent by weight relative to the overall weight of the composition: urea (0.5 wt. %); coenzyme Q10 (0.05 wt. %); polysorbate 20 (0.25 wt. %); 2-phenoxyethanol (0.6 wt. %) and water (q.s.).

Comparative Sample 2B was prepared using the same procedure used in the preparation of Sample 2A, except that urea was not included in the composition. Comparative Sample 2B contained the following components, shown in percent by weight relative to the overall weight of the composition: coenzyme Q10 (0.05 wt. %); polysorbate 20 (0.25 wt. %); 2-phenoxyethanol (0.6 wt. %) and water (q.s.).

Comparative Sample 2C was prepared using the same procedure used in the preparation of Sample 2A, except that coenzyme Q10 was not included in the composition. Comparative Sample 2B contained the following components, shown in percent by weight relative to the overall weight of the composition: urea (0.5 wt. %); polysorbate 20 (0.25 wt. %); 2-phenoxyethanol (0.6 wt. %) and water (q.s.).

Two control samples (Water Blank and Untreated Blank) also were evaluated. The Water Blank contains only water without additional ingredients. The Untreated Blank is a solution of polysorbate 20 (0.25 wt. %) and 2-phenoxyethanol (0.6 wt. %) in water (q.s.).

The testing protocol used in this example is as follows. Five panelists were chosen. The panelists were women between 18 and 49 years of age, and were directed not to use any moisturizers on the arms on the day of the study and throughout the duration of the study. The panelists' arms were washed with soap and dried 15 minutes prior to commencing the study. Three sites on each arm of each panelist were marked off as testing areas. Skin moisturization scores were measured using a Nova DPM 9003 meter. All measurements were done in triplicate and averaged.

Baseline moisturization was measured at the appropriate sites on each arm prior to the application of samples. Each assigned sample was applied (1 ml, applied four times during the first hour) to the appropriate site(s) and triplicate measurements were taken at each site and recorded at 120, 180 and 240 minutes. The results of the cumulative skin moisturizing effect are shown in Table 2. Percent moisturization was calculated based on the Nova DPM 9003 meter readings according to the following equation: Percentage Increase=[(Final Reading-Initial Reading)/Initial Reading]×100.

TABLE 2

| Normalized Data | Initial | 15 min | 30 min | 45 min | 60 min |
|---|---|---|---|---|---|
| Sample 2A | 0.0% | 7.5% | 5.4% | 11.1% | 4.8% |
| Comparative Sample 2B | 0.0% | 1.1% | −2.2% | 3.4% | −2.3% |
| Comparative Sample 2C | 0.0% | −0.3% | 0.1% | 6.5% | 0.8% |

These results demonstrate the improvement in immediate skin moisturizing properties exhibited by a synergistic combination of coenzyme Q10 and urea. Sample A exhibited an 11% maximum increase in skin moisturization over 1 hour, whereas the maximum increase in skin moisturization exhibited by urea alone and coenzyme Q10 alone was 6.5% and 3.4%, respectively.

### EXAMPLE 3

This example demonstrates the cumulative skin moisturizing properties of a synergistic combination of coenzyme Q10 and urea. Three samples (Sample 3A, and Comparative Samples 3B and 3C) were prepared and evaluated. Sample 3A contains a synergistic combination of coenzyme Q10 and urea. Comparative Sample 3B contains coenzyme Q10 in the absence of urea. Comparative Sample 3C contains urea in the absence of coenzyme Q10. Each sample was prepared as an aqueous dispersion containing polysorbate 20 (a nonionic surfactant) and phenoxyethanol (a preservative/antimicrobial agent).

Sample 3A was prepared by dissolving urea and coenzyme Q 10 in a standard aqueous polysorbate 20 solution, diluting the resulting solution with water to form a dispersion of urea and coenzyme Q10, and adding 2-phenoxyethanol. The resulting dispersion contained the following components, shown in percent by weight relative to the overall weight of the composition: urea (0.5 wt. %); coenzyme Q10 (0.05 wt. %); polysorbate 20 (0.25 wt. %); 2-phenoxyethanol (0.6 wt. %) and water (q.s.).

Comparative Sample 3B was prepared using the same procedure used in the preparation of Sample 3A, except that urea was not included in the composition. Comparative Sample 3B contained the following components, shown in percent by weight relative to the overall weight of the composition: coenzyme Q10 (0.05 wt. %); polysorbate 20 (0.25 wt. %); 2-phenoxyethanol (0.6 wt. %) and water (q.s.).

Comparative Sample 3C was prepared using the same procedure used in the preparation of Sample 3A, except that coenzyme Q10 was not included in the composition. Comparative Sample 3B contained the following components, shown in percent by weight relative to the overall weight of the composition: urea (0.5 wt. %); polysorbate 20 (0.25 wt. %); 2-phenoxyethanol (0.6 wt. %) and water (q.s.).

Two control samples (Water Blank and Untreated Blank) also were evaluated. The Water Blank contains only water without additional ingredients. The Untreated Blank is a solution of polysorbate 20 (0.25 wt. %) and 2-phenoxyethanol (0.6 wt. %) in water (q.s.).

The testing protocol used in this example is as follows. Five panelists were chosen. The panelists were women between 18 and 49 years of age, and were directed not to use any moisturizers on the arms on the day of the study and throughout the duration of the study. The panelists' arms were washed with soap and dried 15 minutes prior to commencing the study. Three sites on each arm of each panelist were marked off as testing areas. Skin moisturization scores were measured using a Nova DPM 9003 meter. All measurements were done in triplicate and averaged.

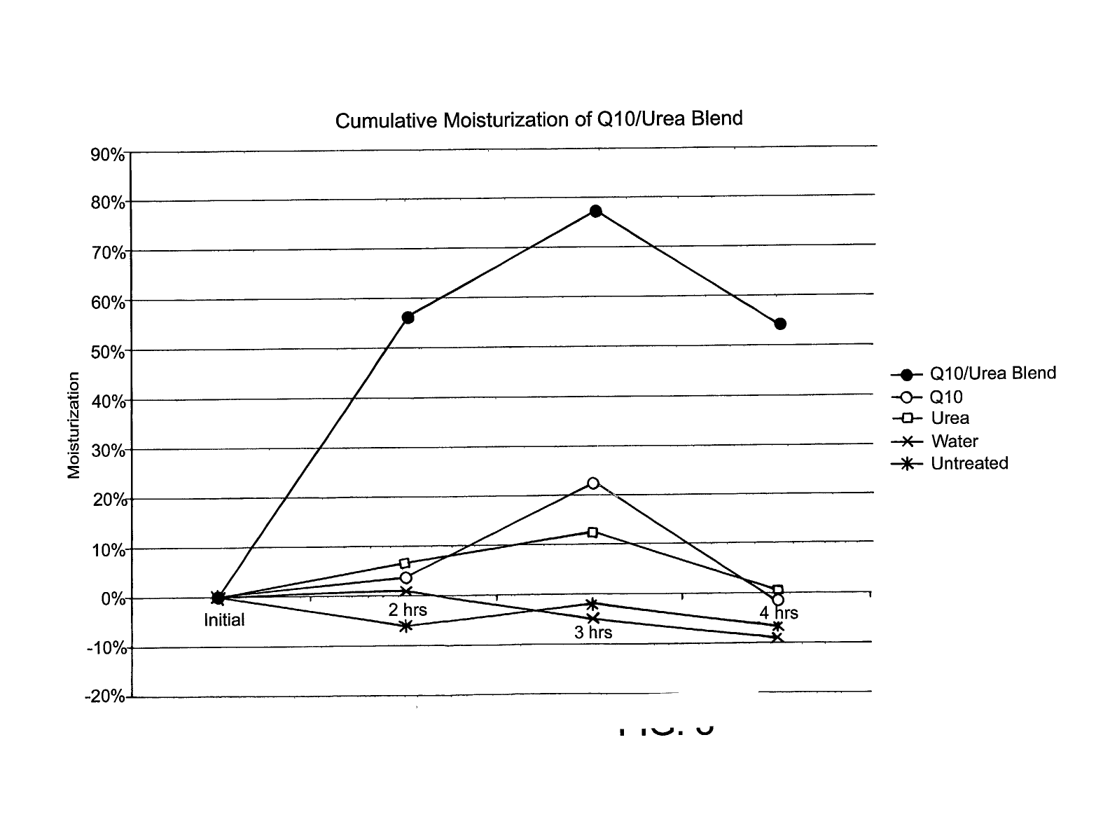

Baseline moisturization was measured at the appropriate sites on each arm prior to the application of samples. Each assigned sample was applied (1 ml, applied four times during the first hour) to the appropriate site(s) and triplicate measurements were taken at each site and recorded at 120, 180 and 240 minutes. The results of the cumulative skin moisturizing effect are shown in Table 3 and are graphically depicted in FIG. 3. Percent moisturization was calculated based on the Nova DPM 9003 meter readings according to the following equation: Percentage Increase [(Final Reading-Initial Reading)/Initial Reading]×100.

TABLE 3

| Product | Initial | 2 hrs | 3 hrs | 4 hrs |
|---|---|---|---|---|
| Urea/Q10 (9 to 1 | 0% | 56% | 78% | 54% |
| ratio) | | | | |
| Q10 | 0% | 4% | 23% | −2% |
| Urea | 0% | 7% | 13% | 0% |
| Water blank | 0% | 1% | −5% | −9% |
| No treatment | 0% | −6% | −2% | −7% |

The foregoing data demonstrate a significant increase in cumulative skin moisturization for the coenzyme Q10/urea blend. The skin moisturization effect exhibited by the coenzyme Q10/urea blend is significantly greater than expected based on the individual moisturization scores for coenzyme Q10 alone and urea alone.

### EXAMPLE 4

This example demonstrates an exemplary composition containing a synergistic combination of coenzyme Q10 and urea. The components of the composition are shown below in Table 4. The quantities of the ingredients shown in the “Qty” column represent the weight of each component in pounds (lb).

TABLE 4

| Component | Qty. (lb) |
|---|---|
| Deionized Water | 79750 |
| Allantoin | 100 |
| Aloe Vera Gel 10x | 100 |
| Caprylic/Capric Triglycerides | 4000 |
| Cetyl Alcohol | 1000 |
| Disodium EDTA | 100 |
| Blend of Botanical Extracts | 100 |
| Acrylates/C10-30 Alkyl Acrylates Cross Polymer | 100 |
| C12-15 Alkyl Octanoate | 2000 |
| Hydrogenated Polyisobutene | 1000 |
| Ethylene Brassylate & PG | 300 |
| Cetearyl Alcohol & Dicetyl Phosphate & Ceteth-10 Phosphate | 3000 |
| Ubiquinone | 50 |
| Glycerin 99.5% | 1000 |
| Glyceryl Monostearate SE | 1500 |
| Methylparaben | 200 |
| Phenoxyethanol | 600 |
| Polysorbate 80 | 250 |
| Propylparaben | 50 |
| Squalane | 250 |
| Stearic Acid Triple Press | 1500 |
| Triethanolamine | 300 |
| Urea | 500 |
| Vitamin E Acetate (USP Grade) | 100 |
| Carbomer 940 | 150 |
| C12-15 Alkyl Benzoate | 1000 |
| Dimethicone 350 Cstk | 1000 |

The composition was prepared as follows. The oil phase was prepared by heating to 80-85° C. the caprylic/capric triglycerides (4000 lb) in a cleaned and sanitized stainless steel oil phase compounding tank. While heating and mixing, the following components were added to the heated caprylic/capric triglycerides: cetearyl alcohol & dicetyl phosphate & ceteth-10 phosphate (3000 lb); C12-15 alkyl octanoate (2000 lb); stearic acid triple press (1500 lb); glyceryl monostearate SE (1500 lb); cetyl alcohol (1000 lb); C12-15 alkyl benzoate (1000 lb); dimethicone (2231 lb) (1000 lb); hydrogenated polyisobutene (1000 lb); squalane (250 lb); and propylparaben (50 lb).

In a separate (main) compounding tank, deionized water was added (64550 lb) and heated to 80-85° C. with mixing. While mixing and heating, the following components are added to the water in the main compounding tank: glycerin 99.5% (1000 lb); urea (500); methylparaben (200 lb); and disodium EDTA (100 lb).

When the batch temperature in the main tank reached 80-85° C., the heating was discontinued and the following components were added: deionized water (12,500 lb); carbomer 940 (150 lb); and acrylates/C10-30 alkyl acrylates cross polymer (100 lb). The components in the main tank were mixed vigorously until all of the carbomer and acrylates were dispersed with no visible lumps, “fish eyes” or undispersed materials and heating was resumed. When the batch temperatures in the main compounding tank and the oil phase compounding tank reached 80-85° C., the oil phase was transferred to the main compounding tank. After flushing the oil phase tank and transferring equipment with deionized water (2450 lb), triethanolamine (300 lb) was added. The combined materials were mixed vigorously for at least 30 minutes, while heating as necessary to maintain a batch temperature of 80-85° C. Thereafter, the mixture in the main compounding tank was cooled so as to reach a batch temperature of 31-36° C.

In a separate stainless steel container, polysorbate 80 (250 lb) and ubiquinone (50 lb) were blended until the ubiquinone appeared evenly dispersed and free from large particulates. When the batch temperature in the main compounding tank cooled to below 50° C., the polysorbate 80/ubiquinone blend was added to the main compounding tank, and the stainless steel container containing the polysorbate 80/ubiquinone blend was rinsed with deionized water (250 lb).

When the batch temperature in the main compounding tank fell below 45° C., the following components were added: phenoxyethanol (600 lb); ethylene brassylate & PG (300 lb); allantoin (100 lb); aloe vera gel (100 lb); vitamin E acetate (100 lb); and blend of botanical extracts (100 lb). Mixing was discontinued when the batch temperature fell below 36° C., and the final product was analyzed and filled into appropriate containers (air diaphragm or positive displacement pump, using 80 mesh filters for all transfer operations). The final product had a pH in the range of 5.0-6.0, and a viscosity of 30,000-70,000 cps (RV, T-Bar “C,” 10 RPM, 60 sec. Penetration).

### EXAMPLE 5

This example demonstrates an exemplary composition containing a synergistic combination of coenzyme Q 10 and urea. Using the procedure described in Example 4, a composition containing the components shown below in Table 5 was prepared. The quantities of the ingredients shown in the “Qty” column represent the weight of each component in pounds (lb).

TABLE 5

| Component | Qty. (lb) |
|---|---|
| Deionized Water | 66740 |
| Allantoin (OTC) | 100 |
| Aloe Vera Gel 10x (OTC) | 100 |
| Disodium EDTA (OTC) | 100 |
| Ethylparaben (OTC) | 100 |
| Glycerin 99.5% (OTC) | 1000 |
| Glyceryl Monostearate SE (OTC) | 1000 |
| Methylparaben (OTC) | 90 |
| Propylparaben (OTC) | 100 |
| Sorbitol 70% | 500 |
| Triethanolamine (OTC) | 320 |
| Urea (OTC) | 300 |
| Vitamin E Oil (OTC) | 100 |
| Oxybenzone (OTC) | 4500 |
| Ethylhexyl p-Methoxycinnamate | 7500 |
| Carbomer 940 (OTC) | 150 |
| Germaben II (OTC) | 1000 |
| C12-15 Alkyl Benzoate (OTC) | 5000 |
| Cyclomethicone (OTC) | 1500 |
| Dimethicone 350 Cstk (OTC) | 250 |
| Blend of Botanical Extracts | 100 |
| Acrylates/C10-30 Alkyl Acrylates Cross Polymer | 100 |
| C12-15 Alkyl Octanoate | 4000 |
| Ethylene Brassylate & PG | 300 |
| Cetearyl Alcohol & Dicetyl Phosphate & Ceteth-10 Phosphate | 2000 |
| Cetyl Alcohol (OTC) | 1500 |
| Stearic Acid (OTC) | 1500 |
| Ubiquinone | 50 |

### EXAMPLE 6

This example demonstrates an exemplary composition containing a synergistic combination of coenzyme Q10 and urea. Using the procedure described in Example 4, a composition containing the components shown below in Table 6 was prepared. The quantities of the ingredients shown in the “Qty” column represent the weight of each component in pounds (lb).

TABLE 6

| Component | Qty. (lb) |
|---|---|
| Octyl Methoxycinnamate | 7500 |
| Methylparaben | 200 |
| Propylparaben USP | 50 |
| DMDM Hydantoin | 500 |
| Disodium EDTA | 100 |
| Allantoin | 10 |
| Dimethicone 350 Cstk | 250 |
| Carbomer 940 | 150 |
| Stearic Acid | 1500 |
| Glycerine 99%, USP | 1000 |
| Cetyl Alcohol | 750 |
| C12-15 Alkyl Benzoate | 3500 |
| Urea | 300 |
| Glyceryl Stearate SE | 1000 |
| Sorbitol 70% | 500 |
| Retinyl Palmitate | 1 |
| Benzophenone-3 | 4500 |
| Triethanolamine 98% | 250 |
| Deionized Water | 71689 |
| Blend of Botanical Extracts | 100 |
| Cyclomethicone | 1000 |
| Acrylates/C10-30 Alkyl Acrylates Cross Polymer | 100 |
| C12-15 Alkyl Octanoate | 1500 |
| Hydrogenated Polyisobutene | 1500 |
| Ethylene Brassylate & PG | 250 |
| Cetearyl Alcohol & Dicetyl Phosphate & Ceteth-10 Phosphate | 1750 |
| Ubiquinone | 50 |

### EXAMPLE 7

This example demonstrates an exemplary composition containing a synergistic combination of coenzyme Q10 and urea. Using the procedure described in Example 4, a composition containing the components shown below in Table 7 was prepared. The quantities of the ingredients shown in the “Qty” column represent the weight of each component in pounds (lb).

TABLE 7

| Component | Qty (lb) |
|---|---|
| Deionized Water | 83,497 |
| Allantoin | 100 |
| Aloe Vera Gel 10X | 100 |
| Ascorbic Acid | 1 |
| Caprylic/Capric Triglycerides | 3000 |
| Cetyl Alcohol | 1000 |
| Retinyl Palmitate | 1 |
| Disodium EDTA | 100 |
| Blend of Botanical Extracts | 100 |
| Acrylates/C10-30 Alkyl Acrylates Cross Polymer | 100 |
| C-12-15 Alkyl Octanoate | 2000 |
| Ethylene Brassylate & PG | 300 |
| Cetearyl Alcohol & Dicetyl Phosphate & Ceteth-10 Phosphate | 1800 |
| Ubiquinone | 50 |
| Glycerin 99.5% | 1000 |
| Glyceryl Monostearate SE | 1500 |
| Methylparaben | 200 |
| Panthenol-DL | 1 |
| Phenoxyethanol | 600 |
| Propylparaben | 100 |
| Squalane | 250 |
| Stearic Acid Triple Press | 1000 |
| Triethanolamine | 300 |
| Urea | 500 |
| Vitamin E Acetate (USP Grade) | 100 |
| Carbomer 940 | 50 |
| C12-15 Alkyl Benzoate | 2000 |

### EXAMPLE 8

This example demonstrates the improved visual appeal, as determined by 20 panelists, of a cosmetic product that includes a synergistic composition of urea and coenzyme Q10. In this example, a group of twenty panelists were chosen and asked to evaluate three cosmetic products and choose one from among them as having the best subjective visual appearance. Three products were evaluated: Comparative Product 8A (containing coenzyme Q10 in the absence of urea), Comparative Product 8B (containing urea in the absence of coenzyme Q10) and Product 8C (containing a synergistic combination of urea and coenzyme Q10). Product 8C was prepared in accordance with Example 4 above. Comparative product 8A was prepared in accordance with Example 4, except that coenzyme Q10 was deleted as a component. Comparative product 8B was prepared in accordance with Example 4, except that urea was deleted as a component. The results of the subjective evaluation are shown in Table 8.

TABLE 8
BEST SUBJECTIVE VISUAL APPEAL

| | | Comparative | Comparative | |
|---|---|---|---|---|
| Panelist | | Product 8A | Product 8B | Product 8C |
| Number | | (Q10 only) | (Urea only) | (Urea & Q10) |
| | 1 | | | x |
| | 2 | | | x |
| | 3 | | x | |
| | 4 | | x | |
| | 5 | | x | |
| | 6 | | | x |
| | 7 | | | x |
| | 8 | | | x |
| | 9 | | x | |
| | 10 | | | x |
| | 11 | | | x |
| | 12 | | | x |
| | 13 | | x | |
| | 14 | | | x |
| | 15 | | | x |
| | 16 | | x | |
| | 17 | | | x |
| | 18 | | | x |
| | 19 | | x | |
| | 20 | | | x |
| | | 0/20 = 0% | 7/20 = 35% | 13/20 = 65% |

The results shown in Table 8 demonstrate that a significantly greater percentage of panelists found Product C to be the most visually appealing relative to Comparative Products A and B. These data demonstrate that a synergistic combination of urea and coenzyme Q10 confers a significant beneficial improvement in product appearance as compared to comparable products containing urea or coenzyme Q10 alone. The improvement can be seen even when relatively small quantities of urea and coenzyme Q10 are present in the composition.

### EXAMPLE 9

This example demonstrates the improvement in subjective rate of absorption (quick absorption) of a cosmetic product that contains a synergistic combination of urea and coenzyme Q 10. In this example, a group of twenty panelists were chosen and asked to subjectively evaluate three cosmetic products in terms of rate of absorption. Three products were evaluated: Comparative Product 9A (containing coenzyme Q10 in the absence of urea), Comparative Product 9B (containing urea in the absence of coenzyme Q10) and Product 9C (containing a synergistic combination of urea and coenzyme Q10). Product 9C was prepared in accordance with Example 4 above. Comparative product 9A was prepared in accordance with Example 4, except that coenzyme Q10 was deleted as a component. Comparative product 9B was prepared in accordance with Example 4, except that urea was deleted as a component. Each product was rated using a rating scale of 1 to 10, with 1 representing the lowest score (poor/slow subjective rate of absorption) and 10 representing the highest score (excellent/high subjective rate of absorption). The results are shown in Table 9.

TABLE 9

| | Comparative | Comparative | Product 9C |
|---|---|---|---|
| Panelist | Product 9A | Product 9B | (Urea & |
| Number | (Q10 only) | (Urea only) | Q10) |
| 1 | 9 | 2 | 6 |
| 2 | 6 | 7 | 8 |
| 3 | 7 | 8 | 8 |
| 4 | 9 | 5 | 10 |
| 5 | 4 | 4 | 6 |
| 6 | 7 | 6 | 7 |
| 7 | 9 | 9 | 10 |
| 8 | 4 | 3 | 4 |
| 9 | 8 | 5 | 5 |
| 10 | 9 | 8 | 4 |
| 11 | 10 | 10 | 9 |
| 12 | 9 | 9 | 10 |
| 13 | 3 | 8 | 9 |
| 14 | 6 | 5 | 5 |
| 15 | 9 | 10 | 10 |
| 16 | 6 | 8 | 7 |
| 17 | 7 | 7 | 4 |
| 18 | 3 | 7 | 9 |
| 19 | 4 | 5 | 7 |
| 20 | 7 | 3 | 9 |
| Average | 6.8 | 6.45 | 7.35 |

These data demonstrate that a synergistic combination of urea and coenzyme Q10 confers a beneficial improvement in subjective rate of absorption as compared to comparable products containing urea or coenzyme Q10 alone.

### EXAMPLE 10

This example demonstrates the improvement in subjective moisturizing properties of a cosmetic product that contains a synergistic combination of urea and coenzyme Q 10. In this example, a group of twenty panelists were chosen and asked to subjectively evaluate three cosmetic products in terms of moisturizing properties. Three products were evaluated: Comparative Product 10A (containing coenzyme Q10 in the absence of urea), Comparative Product 10B (containing urea in the absence of coenzyme Q10) and Product 10C (containing a synergistic combination of urea and coenzyme Q10). Product 10C was prepared in accordance with Example 4 above. Comparative product 10 A was prepared in accordance with Example 4, except that coenzyme Q10 was deleted as a component. Comparative product 10B was prepared in accordance with Example 4, except that urea was deleted as a component. Each product was rated using a rating scale of 1 to 10, with 1 representing the lowest score (poor/slow subjective moisturizing properties) and 10 representing the highest score (excellent subjective moisturizing properties). The results are shown in Table 10.

TABLE 10

| | Comparative | Comparative | |
|---|---|---|---|
| Panelist | Product 10A | Product 10B | Product 10C |
| Number | (Q10 only) | (Urea only) | (Urea & Q10) |
| 1 | 9 | 2 | 6 |
| 2 | 6 | 6 | 6 |
| 3 | 4 | 6 | 6 |
| 4 | 10 | 10 | 6 |
| 5 | 6 | 6 | 7 |
| 6 | 6 | 5 | 6 |
| 7 | 9 | 9 | 9 |
| 8 | 9 | 6 | 7 |
| 9 | 9 | 7 | 7 |
| 10 | 5 | 8 | 8 |
| 11 | 9 | 10 | 10 |
| 12 | 8 | 7 | 10 |
| 13 | 3 | 7 | 8 |
| 14 | 8 | 8 | 7 |
| 15 | 9 | 9 | 8 |
| 16 | 3 | 6 | 5 |
| 17 | 6 | 7 | 8 |
| 18 | 7 | 6 | 7 |
| 19 | 9 | 7 | 6 |
| 20 | 9 | 8 | 10 |
| Average | 7.2 | 7 | 7.35 |

The results shown in Table 10 demonstrate that Product C received the highest average score for subjective moisturizing properties.

### EXAMPLE 11

This example demonstrates the improvement in subjective after-feel properties of a cosmetic product that contains a synergistic combination of urea and coenzyme Q 10. In this example, a group of twenty panelists were chosen and asked to subjectively evaluate three cosmetic products in terms of smooth, silky after-feel. Three products were evaluated: Comparative Product 11A (containing coenzyme Q10 in the absence of urea), Comparative Product 11B (containing urea in the absence of coenzyme Q 10) and Product 11C (containing a synergistic combination of urea and coenzyme Q 10). Product 11C was prepared in accordance with Example 4 above. Comperative product 11A was prepared in accordance with Example 4, except that coenzyme Q10 was deleted as a component. Comparative product 10B was prepared in accordance with Example 4, except that urea was deleted as a component. Each product was rated using a scale of 1 to 10, with 1 representing the lowest score (poor after-feel) and 10 representing the highest score (excellent smooth, silky after-feel). The Table 11.

TABLE 11

| Comparative | Comparative | |
|---|---|---|
| Product 11A | Product 11B | Product 11C |
| (Q10 only) | (Urea only) | (Urea & Q10) |
| 9 | 2 | 6 |
| 5 | 6 | 6 |
| 4 | 5 | 5 |
| 10 | 7 | 7 |
| 4 | 6 | 5 |
| 7 | 5 | 6 |
| 9 | 9 | 10 |
| 7 | 6 | 7 |
| 9 | 6 | 7 |
| 5 | 8 | 3 |
| 9 | 8 | 10 |
| 7 | 8 | 10 |
| 3 | 5 | 8 |
| 7 | 7 | 7 |
| 10 | 9 | 8 |
| 2 | 6 | 4 |
| 7 | 7 | 9 |
| 5 | 8 | 8 |
| 9 | 6 | 6 |
| 7 | 8 | 9 |
| 6.75 | 6.6 | 7.05 |

The results shown in Table 11 demonstrate that Product C received the highest average score in terms of subjective after-feel.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise described. No language in the specification should be construed as indicating that any non-essential element is essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context.

What is claimed is:

1. A cosmetic composition comprising urea and coenzyme Q10, wherein the ratio of urea to coenzyme Q10 in the composition is from about 1:5 to about 20:1 (wt./wt.).

2. The composition of claim 1, wherein the composition is in the form of an emulsion.

3. The composition of claim 2, wherein the emulsion is an oil-in-water or Water-in-oil emulsion.

4. The composition of claim 1, further comprising water, which is present in an amount of from about 50% to about 90% by weight of the composition.

5. The composition of claim 3, further comprising oil, which is present in an amount of from about 10% to about 50% by weight of the composition.

6. The composition of claim 1, wherein the urea is present in an amount of from about 0.02% to about 15% by weight of the composition.

7. The composition of claim 6, wherein the urea is present in an amount of from about 0.1% to about 10% by weight of the composition.

8. The composition of claim 1, wherein the coenzyme Q 10 is present in an amount of from about 0.001% to about 5% by weight of the composition.

9. The composition of claim 8, wherein the coenzyme Q10 is present in an amount of from about 0.01% to about 2% by weight of the composition.

10. The composition of claim 1, wherein the ratio of urea to coenzyme Q 10 in the composition is at least about 3:1 (wt./wt.).

11. The composition of claim 1, wherein the ratio of urea to coenzyme Q 10 in the composition is at least about 6:1 (wt./wt.).

12. The composition of claim 1, further comprising an emulsifier.

13. The composition of claim 12, wherein the emulsifier is present in an amount of from about 1.5% to about 20% by weight of the composition.

14. The composition of claim 1, further comprising a thickener.

15. The composition of claim 14, wherein the thickener is selected from the group consisting of gums, cellulosics, acrylic polymers, carbomers, and combinations hereof.

16. The composition of claim 14, wherein the thickener is present in an amount of from about 0.1% to about 5% by weight of the composition.

17. The composition of claim 1, further comprising a sunscreen.

18. The composition of claim 17, wherein the sunscreen is present in an amount of from about 0.01% to about 20% by weight of the composition.

19. The composition of claim 1, further comprising a preservative.

20. The composition of claim 1, wherein the oil phase comprises an oil selected from the group consisting of silicone oils, triglyceride esters, natural oils, waxes, hydrocarbons, phospholipids, polyhydric fatty alcohols, polyether derivatives, and combinations thereof.

21. The composition of claim 1, further comprising a solid emollient.

22. The composition of claim 21, wherein the solid emollient is present in an amount of from about 0.1% to about 10% by weight of the composition.

23. The composition of claim 1, further comprising a soothing additive.

24. The composition of claim 23, wherein the soothing additive is selected from the group consisting of allantoin, natural moisturizing and soothing botanical extracts, anti-inflammatory agents, and combinations thereof.

25. The composition of claim 23, wherein the soothing additive is present in an amount of from about 0.001% to about 5% by weight of the composition.

26. The composition of claim 1, wherein the composition further comprises an antioxidant other than coenzyme Q 10.

27. The composition of claim 26, wherein the antioxidant is selected from the group consisting of antioxidant vitamins and combinations thereof.

28. The composition of claim 26, wherein the antioxidant is present in an amount of from about 0.001% to about 5% by weight of the composition.

29. The composition of claim 1, further comprising a chemical neutralizer.

30. The composition of claim 29, wherein the neutralizer comprises one or more chemical bases.

31. The composition of claim 29, wherein the neutralizer is present in an amount of from about 0.01% to about 10% by weight of the composition.

32. The composition of claim 1, further comprising an odor-masking agent.

33. The composition of claim 32, wherein the odor masking agent is present in an amount of from about 0.01% to about 3% by weight of the composition.

34. The composition of claim 1, further comprising a moisturizing additive.

35. The composition of claim 34, wherein the moisturizing additive is selected from the group consisting of esters, humectants, natural botanical extracts, phospholipids, silicones, occlusive agents, natural oils, and combinations thereof.

36. The composition of claim 35, wherein the moisturizing additive is present in an amount of from about 0.0001% to about 25% by weight of the composition.

37. The composition of claim 1, wherein the composition imparts skin with a moisture index, as measured by Novameter DPM9003, of at least about 50% relative to untreated skin when applied in an amount of 155 mg/cm² skin about 120 minutes after application.

38. The composition of claim 1, wherein the composition is in the form a cream, a lotion, a solution, an ointment, or a gel.

39. A cosmetic composition comprising:
(a) urea, which is present in an amount of from about 0. 1% to about 10% by weight of the composition;
(b) coenzyme Q10, which is present in an amount of from about 0.001% to about 5% by weight of the composition; and, optionally, one or more of the following ingredients:
(i) an emulsifier;
(ii) a thickener;
(iii) a preservative;
(iv) a sunscreen;
(v) a chemical neutralizer;
(vi) an odor-masking agent;
(vii) a solid emollient;
(viii) a soothing additive;
(ix) a moisturizing additive; and
(x) an antioxidant.

40. The composition of claim 39, further comprising water and oil.

41. A cosmetic composition comprising an emulsion, the emulsion comprising:
(a) an aqueous phase;
(b) an oil phase;
(c) urea, which is present in an amount of from about 0.1% to about 10% by weight of the composition;
(d) coenzyme Q10, which is present in an amount of from about 0.001% to about 5% by weight of the composition; and, optionally, one or more of the following ingredients:
(i) an emulsifier;
(ii) a thickener;
(iii) a preservative;
(iv) a sunscreen;
(v) a chemical neutralizer;
(vi) an odor-masking agent;
(vii) a solid emollient;
(viii) a soothing additive;
(ix) a moisturizing additive; and
(x) an antioxidant.

42. The composition of claim 40, wherein:
the aqueous phase is present in an amount of from about 50% to about 90% by weight of the composition;
the oil phase is present in an amount of from about 10% to about 50% by weight of the composition;
the emulsifier is present in an amount of from about 0% to about 20% by weight of the composition;
the thickener is present in an amount of from about 0% to about 5% by weight of the composition;
the preservative is present in an amount of from about 0.01% to about 3% by weight of the composition;
the sunscreen is present in an amount of from about 0% to about 20% by weight of the composition;
the chemical neutralizer is present in an amount of from about 0% to about 10% by weight of the composition;
the odor-masking agent is present in an amount of from about 0% to about 3% by weight of the composition;
the solid emollient is present in an amount of from about 0% to about 10% by weight of the composition;
the soothing additive is present in an amount of from about 0% to about 5% by weight of the composition;
the moisturizing additive is present in an amount of from about 0% to about 20% by weight of the composition; and
the antioxidant is present in an amount of from about 0% to about 1% by weight of the composition.

43. A method of enhancing moisture retention in the skin from the epidermis, the method comprising topically applying a moisture-retaining inhibiting effective amount of the composition of claim 1 to the skin.

44. A method reducing the rate of escape of water from the epidermis, the method comprising topically applying a moisture-loss inhibiting effective amount of the composition of claim 40 to the skin.

45. A method of improving the after-feel of a cosmetic composition when the composition is applied to the skin, the method comprising including in the composition an after-feel enhancing-effective amount of coenzyme Q10 and urea, wherein the ratio of urea to coenzyme Q10 in the composition is from about 1:5 to about 20:1 (wt./wt.).
